(19) United States
(12) Patent Application Publication
(10) Pub. No.: US 2015/0232393 A1
(43) Pub. Date: Aug. 20, 2015

(54) PROCESS FOR THE PURIFICATION OF A CYCLOHEXANE AIR OXIDATION PRODUCT STREAM

(71) Applicant: David Lee Valdez, Victoria, TX (US)

(72) Inventor: David Lee Valdez, Victoria, TX (US)

(73) Assignee: INVISTA NORTH AMERICA S.A R.L., Wilmington, DE (US)

(21) Appl. No.: 14/415,159

(22) PCT Filed: Jul. 19, 2012
(86) PCT No.: PCT/US2012/047384

§ 371 Date: Apr. 30, 2015

(51) Int. Cl. C07B 63/00; C07C 407/00; C07C 7/10; C07C 49/403; C07C 29/86

(52) U.S. Cl. CPC C07B 63/00; C07C 49/403; C07C 29/86; C07C 7/10; C07C 407/003; C07C 2101/14

(57) ABSTRACT

A method is disclosed for removing contaminants from a feed stream to a hydrogenation process that begins with providing a product mixture from an air oxidation reaction. A first liquid separation process and cooling procedure is used on the product mixture to form a cooled product mixture and a first vapor stream. The cooled product mixture is then subjected to a water wash to form a washed product mixture and an aqueous exit stream, wherein a majority of the water soluble other oxidation products from the cooled product mixture are present in the aqueous exit stream. Next, the washed product stream undergoes a second liquid separation and water removal to form a treated product mixture and a second vapor stream. Finally, the treated product mixture is recovered and can be fed to a hydrogenation process.

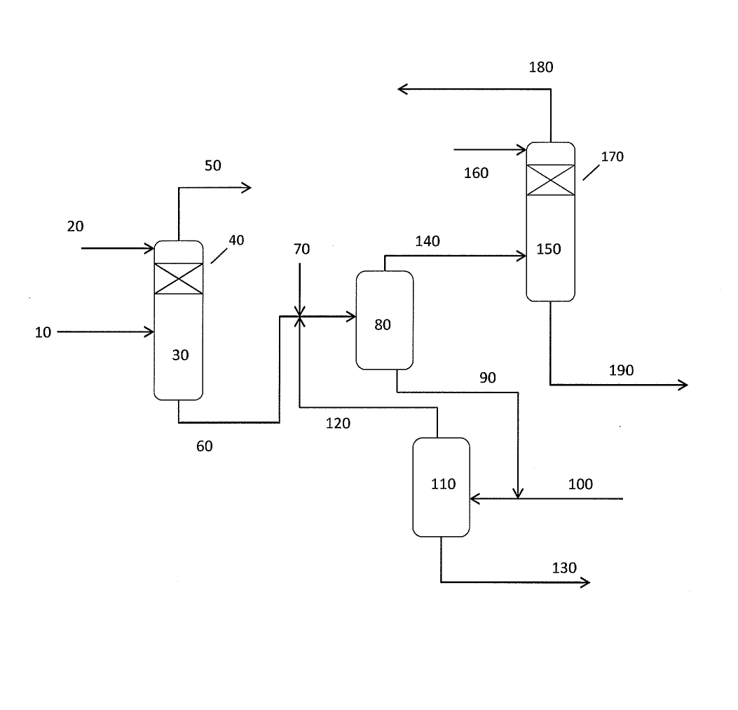

### FIELD OF THE INVENTION

This disclosure relates to a method for treating a feed stream to a hydrogenation process. More specifically, it relates to improving the yield of a cyclohexylhydroperoxide hydrogenation process by decreasing the amount of reactants lost during the treatment of the feed stream.

### BACKGROUND OF THE INVENTION

The air oxidation of cyclohexane is an important process for the production of caprolactam and adipic acid, which are used in the manufacture of synthetics such as nylon. The oxidation of cyclohexane by air produces a reaction product comprising cyclohexanol (A), cyclohexanone (K) cyclohexylhydroperoxide (CHHP) and small amounts of by-products. Cyclohexanone (K) and cyclohexanol (A) are the main product of the overall process and the mixture is commonly known as KA oil. Several patents, herein incorporated by reference, such as U.S. Pat. Nos. 3,530,185, 3,957,867, 5,780,683 and 6,703,529 teach the preparation of a mixture containing cyclohexanol, cyclohexanone and cyclohexylhydroperoxide by the air oxidation of cyclohexane.

It is well known that cyclohexylhydroperoxide in a mixture containing cyclohexanol, cyclohexanone, other products of the air oxidation reactions will react to form cyclohexanol and cyclohexanone. However, this process does not result in a high yield of KA oil and other waste materials are formed. It has been found that the highest yields of KA oil can be achieved when the oxidation of cylcohexane is performed under conditions that result in a greater amount of cyclohexylhydroperoxide and the cyclohexylhydroperoxide is then treated by hydrogenation in a separate process to cyclohexanone (K) and cyclohexanol (A) to give an increased overall yield of KA oil. For example, the preparation of cyclohexanol and cyclohexanone from cyclohexylhydroperoxide by hydrogenation has been in described in U.S. Pat. Nos. 3,694,511 and 3,927,108, herein incorporated by reference.

If a mixture containing cyclohexylhydroperoxide and a cobalt catalyst is subjected to a hydrogenation reaction in the presence of a fixed bed hydrogenation catalyst, cyclohexanone and cyclohexanol are among the products produced, but the reactor soon becomes fouled with cobalt containing residues and with residues from other oxidation products produced during the initial oxidation reaction, i.e., diacids and hydroxy acids, and the reaction slows and the yield of desired product is reduced. Several patents, herein incorporated by reference, such as U.S. Pat. Nos. 3,927,108 and 3,923,895 teach treating the product stream from the oxidation reaction to remove residual catalyst and other oxidation products prior to hydrogenation.

U.S. Pat. No. 4,720,592, herein incorporated by reference, describes a process that reduces this catalyst fouling, wherein the product of a cyclohexane oxidation process containing cobalt and an organic phosphate ester is extracted with water and hydrogenated in a reactor containing a palladium catalyst on a silica substrate. However, treating the product stream to remove the cobalt catalyst and other oxidation by-products also results in the loss of cyclohexylhydroperoxide. This loss of cyclohexylhydroperoxide results in a reduced yield of KA oil from the hydrogenation process.

Therefore, there is a need for a process for treating a product stream from a cyclohexane oxidation reaction to remove residual catalyst and unwanted oxidation by-products, while retaining cyclohexylhydroperoxide in the product stream.

### SUMMARY OF THE INVENTION

The present invention relates to a process for treating a product stream from a cyclohexane oxidation reaction to remove residual catalyst and unwanted oxidation by-products. During the process, the loss of cyclohexylhydroperoxide is minimized. An embodiment of the present invention comprises the steps of:
(a) providing a product mixture from an air oxidation reaction comprising of desired products, dissolved gases, and other oxidation products;
(b) cooling the product mixture of step (a) in a first liquid separation process to form a cooled product mixture and a first vapor stream, wherein about 98 wt % to about 99.5 wt % of the dissolved gases from the product mixture of step (a) are present in the first vapor stream and greater than 98 wt % of the desired products from the product mixture of step (a) are present in the cooled product mixture;
(c) contacting the cooled product mixture of step (b) with water to form a washed product mixture and an aqueous exit stream, wherein a majority of the water soluble other oxidation products from the cooled product mixture of step (b) are present in the aqueous exit stream;
(d) removing water from the washed product mixture of step (c) in a second liquid separation process to form a treated product mixture and a second vapor stream, wherein greater than 98 wt % of the desired products from the washed product mixture of step (c) are present in the treated product mixture; and
(e) recovering the treated product mixture of step (d), wherein the treated product mixture is suitable as a feed stream for the hydrogenation process.

In another embodiment, the air oxidation reaction is the air oxidation of cyclohexane.

In another embodiment, the product mixture comprises cyclohexylhydroperoxide (CHHP), cyclohexanone, cyclohexanol, cyclohexane, other oxidation products and organic ester which is soluble in the mixture and having the formula:

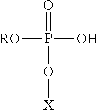

Where R is selected from the group consisting of C4-C12 alkyl radicals and C5-C8 cycloalkyl radicals, and X is H or R.

In another embodiment, the desired products comprise CHHP, cyclohexanone and cyclohexanol.

In another embodiment, the other oxidation products comprise residual catalyst, diacids, monoacids and hydroxyacids.

In another embodiment, the residual catalyst is a cobalt catalyst selected from the group consisting of cobalt naphthenate, cobalt octoate, cobalt laurate, cobalt palminate, cobalt stearate, cobalt linoleate, cobalt acetylacetonate and combinations thereof.

In another embodiment, the amount of organic phosphate ester in the product mixture is present in a molar ratio to cobalt of 3:1 to 50:1.

In another embodiment, step (b) is carried out in a flash cooler and the liquid separation is accomplished with a cylcohexane stream which contacts the first vapor stream from step (a) in a vapor-liquid contacting zone in the flash cooler.

In another embodiment, the vapor-liquid contacting zone comprises sprays, trays or packing in the flash cooler.

In another embodiment, step (b) is carried out at a temperature that minimizes the thermal decomposition of CHHP.

In another embodiment, the flash cooling takes place at a temperature range of about 100° C. to about 140° C.

In another embodiment, the dissolved gas is nitrogen.

In another embodiment, the aqueous exit stream of step (c) is contacted with an extractant to form a treated aqueous exit stream, wherein the extractant recovers from about 60 wt % to about 90 wt % of the desired products from the aqueous exit stream of step (c).

In another embodiment, the extractant is cylcohexane.

In another embodiment, the treated aqueous exit stream is mixed with the cooled product mixture of step (b) prior to step (c).

In another embodiment, step (d) is carried out in a water flasher and the vapour-liquid extraction is accomplished with a cylcohexane stream which contacts the washed product mixture of step (c) in a vapor-liquid contacting zone in the water flasher.

In another embodiment, the vapor-liquid contacting zone comprises sprays, trays or packing in the water flasher.

### BRIEF DESCRIPTION OF THE DRAWING

The FIGURE is a process diagram for an embodiment of the present invention.

### DETAILED DESCRIPTION OF THE INVENTION

The present invention relates to a process for treating a product stream from a cyclohexane oxidation reaction to remove residual catalyst and unwanted oxidation by-products. During the process, the loss of cyclohexylhydroperoxide is minimized.

All patents, patent applications, test procedures, priority documents, articles, publications, manuals, and other documents cited herein are fully incorporated by reference to the extent such disclosure is not inconsistent with this invention and for all jurisdictions in which such incorporation is permitted.

Referring to the FIGURE, an exemplary embodiment of the present invention is herein described. The FIGURE is a process diagram process for treating a product stream (10) from a cylcohexane oxidation reaction (not shown) prior to being hydrogenated. The product stream may contain cyclohexane, cyclohexanol, cyclohexanone, cyclohexylhydroperoxide (CHHP) and other products of the oxidation of cyclohexane including diacids, monoacids and hydroxyacids. The mixture of cyclohexanol and cyclohexanone is also referee to as KA oil. A process for treating a product stream from a cylcohexane oxidation reaction taught in U.S. Pat. No. 4,720,592, incorporated herein by reference. The mixture may also contain a cobalt catalyst which is soluble in the mixture. The mixture may contain an organic phosphate ester which is soluble in the mixture. The organic phosphate ester may be added to an air oxidation reactor or to the mixture leaving an air oxidation reactor.

The air oxidation of cyclohexane with a soluble cobalt catalyst has been taught in U.S. Pat. No. 3,957,876, which is herein incorporated by reference. Suitable catalysts include cobalt naphthenate, cobalt octoate, cobalt laurate, cobalt palminate, cobalt stearate, cobalt linoleate, cobalt acetylacetonate and combinations thereof.

Suitable organic phosphate esters have the formula:

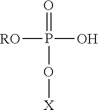

where R is selected from the group consisting of C4-C2 alkyl radicals and C5-C8 cycloalkyl radicals, and X is H or R. An example of a commercially available organic phosphate ester is Emphos PS-400, which contains phosphoric acid, mono(2-thylhexyl)phosphoric acid and di(2-ethylhexyl)phosphoric acid. If a cobalt catalyst is also present in the product stream (10), the amount of organic phosphate ester present in the mixture should exceed on a molar basis the amount of cobalt catalyst present in the mixture, and preferably the molar ratio of organic phosphate to cobalt is in the range of 3:1 to 50:1.

The product stream (10) is sent to a flash cooler (30) to remove dissolved gases such as nitrogen and to quickly drop the product stream temperature so as to minimize the thermal decomposition of cyclohexanol, cyclohexanone and CHHP (“the desired products”). The desired products are volatile and a significant portion may be lost to the flash cooler vapor stream (50). To minimize the loss of the desired products in the flash cooler (30), this step is carried out in a vessel utilizing a reflux stream (20) and vapor-liquid contacting (40). In an exemplary embodiment of the present invention, reflux stream comprises cylcohexane. The vapor-liquid contacting (40) comprises sprays, trays or packing in the flash cooler (30) above the feed point. The sprays, trays or packing retain the desired products in the product stream, and this stream leaves the flash cooler (30) as cooled product stream (60). The vapor stream (50) leaving the flash cooler (30) will contain from about 98 wt % to about 99.5 wt % of the dissolved gases from the product stream (10). The cooled product stream (60) leaving the flash cooler (30) will contain from greater than 98 wt % of the desired products from the product stream (10).

The cooled product stream (60) is sent to decanter (80) to be extracted with water (70) to remove a substantial portion of the other oxidation products that are water soluble and the cobalt catalyst if present. The other oxidation products comprise diacids, monoacids and hydroxyacids. In particular embodiments of the present invention, the other oxidation products may comprise 6-hydroxyl caproic acid, 5-hydroxy valeric acid, succinic acid, adipic acid and formic acid. The washed product stream (140) leaving the decanter (80) will contain a majority of the desired products from the treated product stream. In other embodiments of the current invention, the water extraction may be achieved with a series of decanters or a single fixed bed extractor may be employed.

Because the cyclohexanol, cyclohexanone and CHHP are also water soluble, a portion of the desired product may be lost into the aqueous exit stream (90) leaving the decanter (80). In an exemplary embodiment of the current invention, the aqueous exit stream (90) is sent to decanter (110) and extracted with cyclohexane stream (100). In other embodiments, any suitable solvent may be used to extract the desired products. The desired products leave the decanter (110) in treated aqueous exit stream 120 which is combined with the treated product stream (60) prior to being fed to decanter (80). Preferably, from about 60 wt % to about 90 wt % of the desired products from the aqueous exit stream (90) will be recovered in the treated aqueous exit stream (120). The aqueous waste stream (130) may be sent to a waste water facility for treatment.

In another embodiment of the current invention (not shown), the aqueous exit stream (90) may be sent to a refining section of the process so that cyclohexanol and cyclohexanone are recovered. The CHHP dissolved in the water will eventually be thermally decomposed to cyclohexanol and cyclohexanone in the refining section.

After extraction, the washed water stream (140) is sent to water flasher (150) to dehydrate the stream prior to being fed to the hydrogenation process. The desired products are volatile and a significant portion may be lost to the water flasher vapor stream (180). To minimize the loss of the desired products in the water flasher (150), this step is carried out in a vessel utilizing a reflux stream (160) and vapor-liquid contacting (170). In an exemplary embodiment of the present invention, reflux stream (160) comprises cylcohexane. The vapor-liquid contacting (170) comprises sprays, trays or packing in the flash cooler (150) above the feed point. The sprays, trays or packing retain the desired products in the product stream, and this stream leaves the water flasher (150) as treated product stream (190). The treated product stream (190) leaving the flash cooler (150) will contain greater than 98 wt % of the desired products from the washed water stream (140). The treated product stream (190) is recovered and can be sent to a hydrogenation process.

### EXAMPLES

The following Examples demonstrate the present invention and its capability for use. The invention is capable of other and different embodiments, and its several details are capable of modifications in various apparent respects, without departing from the scope and spirit of the present invention. Accordingly, the Examples are to be regarded as illustrative in nature and non-limiting.

### Comparative Example 1

U.S. Pat. No. 4,720,592 teaches a method of treating a feed stream to a CHHP hydrogenation process. A method of reducing catalyst fouling in a subsequent hydrogenation process is achieved by treating a cyclohexane oxidation tails stream via a flash cooler, a water wash and a water flasher. The resulting hydrogenation feed stream contains 1.2 wt % CHHP, 0.886 wt % Cyclohexanone and 2.32 wt % cyclohexanol. As a result of the process steps intended to reduce catalyst fouling, over 80 wt % of the CHHP exiting the cyclohexane oxidizer was maintained in the hydrogenation feed stream.

### Example 1

The current invention improves upon the method taught in U.S. Pat. No. 4,720,592 by inserting process steps described above to minimize the loss of CHHP prior to hydrogenation. The flash cooling and water flashing steps of the process were conducted at INVISTA's Victoria plant. Following a cyclohexane oxidation process, the oxidizer tails stream contained 2.2 wt % CHHP, 0.6 wt % cyclohexanone and 1.4% cyclohexanol. The flash cooling step was performed with a cyclohexane extraction process to recover CHHP that would be lost in the process. The resulting hydrogenation feed stream contained 2.8 wt % CHHP, 0.8 wt % cyclohexanone and 1.8% cyclohexanol. Over 83.5 wt % of the CHHP from the oxidizer tails was maintained in the hydrogenation feed stream. As described in the process description above, additional CHHP may be recovered by operating the water washing steps with solvent extraction steps. At INVISTA's Wilton plant, the wash water feed to the solvent extraction process contained 0.7 wt % CHHP, 0.1% wt % cyclohexanone and 0.2 wt % cyclohexanol. After the extraction process, the wash water stream contained 0.01 wt % CHHP, 0.01% wt % cyclohexanone and 0.02 wt % cyclohexanol. Theoretical modeling data shows that an additional 0.8% of the total CHHP, cyclohexanone and cyclohexanol produced by the air oxidation of cyclohexane may be recovered via cyclohexane extraction during the water washing steps. As a result, between 84 to 85 wt % of the CHHP leaving the oxidizer tails can be retained in the hydrogenation feed stream using the process of the current invention.

### Example 2

The present example is a method for removing contaminants from a feed stream to a hydrogenation process that begins with providing a product mixture from an air oxidation reaction comprising of desired products, dissolved gases and other oxidation products. A first liquid separation process and cooling procedure is used on the product mixture to form a cooled product mixture and a first vapor stream, wherein about 98 wt % to about 99.5 wt % of the dissolved gases from the product mixture are present in the first vapor stream and greater than 98 wt % of the desired products from the product mixture are present in the cooled product mixture. The cooled product mixture is then subjected to a water wash to form a washed product mixture and an aqueous exit stream, wherein a majority of the water soluble other oxidation products from the cooled product mixture are present in the aqueous exit stream. Next, the washed product stream undergoes a second liquid separation and water removal to form a treated product mixture and a second vapor stream, wherein greater than 98 wt % of the desired products from the washed product mixture of are present in the treated product mixture. Finally, the treated product mixture is recovered and can be fed to a hydrogenation process.

### Example 3

The process of Example 2 is repeated with additional steps. In this example, the air oxidation reaction is the air oxidation of cyclohexane.

### Example 4

The process of Example 3 is repeated with additional steps. In this example, the product mixture comprises cyclohexylhydroperoxide (CHHP), cyclohexanone, cyclohexanol, cyclohexane, other oxidation products and organic ester which is soluble in the mixture and having the formula:

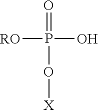

Where R is selected from the group consisting of C4-C12 alkyl radicals and C5-C8 cycloalkyl radicals, and X is H or R.

### Example 5

The process of Example 4 is repeated with additional steps. In this example, the desired products comprise CHHP, cyclohexanone and cyclohexanol.

### Example 6

The process of Example 5 is repeated with additional steps. In this example, the other oxidation products comprise residual catalyst, diacids, monoacids and hydroxyacids.

### Example 7

The process of Example 6 is repeated with additional steps. In this example, the residual catalyst is a cobalt catalyst selected from the group consisting of cobalt naphthenate, cobalt octoate, cobalt laurate, cobalt palminate, cobalt stearate, cobalt linoleate, cobalt acetylacetonate and combinations thereof.

### Example 8

The process of Example 7 is repeated with additional steps. In this example, the amount of organic phosphate ester in the product mixture is present in a molar ratio to cobalt of 3:1 to 50:1.

### Example 9

The process of Example 8 is repeated with additional steps. In this example, the first liquid separation of Example 2 is carried out in a flash cooler and the liquid separation is accomplished with a cylcohexane stream which contacts the first vapor stream in a vapor-liquid contacting zone in the flash cooler.

### Example 10

The process of Example 9 is repeated with additional steps. In this example, the vapor-liquid contacting zone comprises sprays, trays or packing in the flash cooler.

### Example 11

The process of Example 11 is repeated with additional steps. In this example, the flash cooling is carried out at a temperature that minimizes the thermal decomposition of CHHP.

### Example 12

The process of Example 11 is repeated with additional steps. In this example, wherein flash cooling is carried out at a temperature range of about 100° C. to about 140° C.

### Example 13

The process of Example 12 is repeated with additional steps. In this example, where in the dissolved gas is nitrogen.

### Example 14

The process of Example 2 is repeated with additional steps. In this example, wherein the aqueous exit stream is contacted with an extractant to form a treated aqueous exit stream, wherein the extractant recovers from about 60 wt % to about 95 wt % of the desired products from the aqueous exit stream.

### Example 15

The process of Example 14 is repeated with additional steps. In this example, the extractant is cylcohexane.

### Example 16

The process of Example 15 is repeated with additional steps. In this example, the treated aqueous exit stream is mixed with the cooled product mixture prior to the water wash.

### Example 17

The process of Example 16 is repeated with additional steps. In this example, wherein second liquid separation of Example 2 is carried out in a water flasher and the vapor-liquid extraction is accomplished with a cylcohexane stream which contacts the washed product mixture in a vapor-liquid contacting zone in the water flasher.

### Example 18

The process of Example 17 is repeated with additional steps. In this example, the vapor-liquid contacting zone comprises sprays, trays or packing in the water flasher.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also the individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±8%, or ±10%, of the numerical value(s) being modified. In addition, the phrase “about ‘x’ to ‘y’” includes “about ‘x’ to about ‘y’”.

While the illustrative embodiments of the invention have been described with particularity, it will be understood that the invention is capable of other and different embodiments and that various other modifications will be apparent to and may be readily made by those skilled in the art without departing from the spirit and scope of the invention. Accordingly, it is not intended that the scope of the claims hereof be limited to the examples and descriptions set forth herein but rather that the claims be construed as encompassing all the features of patentable novelty which reside in the present disclosure, including all features which would be treated as equivalents thereof by those skilled in the art to which the invention pertains.

What is claimed:

1. A method for treating a feed stream to a hydrogenation process, comprising the steps of:
(a) providing a product mixture from an air oxidation reaction comprising of desired products, dissolved gases, and other oxidation products;
(b) cooling the product mixture of step (a) in a first liquid separation process to form a cooled product mixture and a first vapor stream, wherein about 98 wt % to about 99.5 wt % of the dissolved gases from the product mixture of step (a) are present in the first vapor stream and greater than 98 wt % of the desired products from the product mixture of step (a) are present in the cooled product mixture;
(c) contacting the cooled product mixture of step (b) with water to form a washed product mixture and an aqueous exit stream, wherein a majority of the water soluble other oxidation products from the cooled product mixture of step (b) are present in the aqueous exit stream;
(d) removing water from the washed product mixture of step (c) in a second liquid separation process to form a treated product mixture and a second vapor stream, wherein greater than 98 wt % of the desired products from the washed product mixture of step (c) are present in the treated product mixture; and
(e) recovering the treated product mixture of step (d), wherein the treated product mixture is suitable as a feed stream for the hydrogenation process.

2. The method of claim 1 wherein the air oxidation reaction is the air oxidation of cyclohexane.

3. The method of claim 2 wherein the product mixture comprises cyclohexylhydroperoxide (CHHP), cyclohexanone, cyclohexanol, cyclohexane, other oxidation products and organic ester which is soluble in the mixture and having the formula: 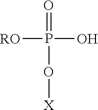
Where R is selected from the group consisting of C₄-C₁₂ alkyl radicals and C₅-C₈ cycloalkyl radicals, and X is H or R.

4. The method of claim 3 wherein the desired products comprise CHHP, cyclohexanone and cyclohexanol.

5. The method of claim 3 wherein the other oxidation products comprise residual catalyst, diacids, monoacids and hydroxyacids.

6. The method of claim 3 wherein the residual catalyst is a cobalt catalyst selected from the group consisting of cobalt naphthenate, cobalt octoate, cobalt laurate, cobalt palminate, cobalt stearate, cobalt linoleate, cobalt acetylacetonate and combinations thereof.

7. The method of claim 6 wherein the amount of organic phosphate ester in the product mixture is present in a molar ratio to cobalt of 3:1 to 50:1.

8. The method of claim 6 wherein step (b) is carried out in a flash cooler and the liquid separation is accomplished with a cylcohexane stream which contacts the first vapor stream from step (a) in a vapor-liquid contacting zone in the flash cooler.

9. The method of claim 8 wherein the vapor-liquid contacting zone comprises sprays, trays or packing in the flash cooler.

10. The method of claim 8 wherein step (b) is carried out at a temperature that minimizes the thermal decomposition of CHHP.

11. The method of claim 10 wherein the flash cooling takes place at a temperature range of about 100° C. to about 140° C.

12. The method of step 8 where in the dissolved gas is nitrogen.

13. The method of claim 6 wherein the aqueous exit stream of step (c) is contacted with an extractant to form a treated aqueous exit stream, wherein the extractant recovers from about 60 wt % to about 95 wt % of the desired products from the aqueous exit stream of step (c).

14. The method of claim 13 wherein the extractant is cylcohexane.

15. The method of claim 13 wherein the treated aqueous exit stream is mixed with the cooled product mixture of step (b) prior to step (c).

16. The method of claim 6 wherein step (d) is carried out in a water flasher and the vapor-liquid extraction is accomplished with a cylcohexane stream which contacts the washed product mixture of step (c) in a vapor-liquid contacting zone in the water flasher.

17. The method of claim 16 wherein the vapor-liquid contacting zone comprises sprays, trays or packing in the water flasher.
